(19) United States
(12) Patent Application Publication
(10) Pub. No.: US 2025/0370146 A1
(43) Pub. Date: Dec. 4, 2025

(54) DETERMINATION OF ACTINIUM-225 IN URINE

(71) Applicant: Atomic Energy Of Canada Limited / Énergie Atomique Du Canada Limitée, Chalk River (CA)

(72) Inventor: Nicolas Guérin, Chalk River (CA)

(21) Appl. No.: 18/875,409

(22) PCT Filed: Jun. 16, 2023
(86) PCT No.: PCT/CA2023/050834

§ 371 Date: Dec. 16, 2024

Related U.S. Application Data
(60) Provisional application No. 63/352,663, filed on Jun. 16, 2022.

(51) Int. Cl. G01T 1/167; G01N 1/34; G01N 1/40; G01N 33/84; G01T 7/02

(52) U.S. Cl. CPC G01T 1/167; G01N 1/34; G01N 1/4044; G01N 1/4077; G01N 33/84; G01T 7/02; G01N 2001/4088

(57) ABSTRACT

Methods of determining actinium-225 in a human urine sample can be used to establish the effective dose received by persons working with or otherwise exposed to this isotope. The human urine sample can be prepared to produce a pre-concentrated sample, which can include co-precipitating Ac and separating a first precipitate. At least some organic matter can be broken down in the pre-concentrated sample to produce a decomposed sample, which can include adding an oxidative agent to the pre-concentrated sample. The decomposed sample can be purified to produce a measurement sample, which can include passing the decomposed sample through a purification media and eluting actinium from the purification media.

### CROSS-REFERENCE TO RELATED APPLICATION

This application claims priority to U.S. Provisional Application No. 63/352,663 filed on Jun. 16, 2022, the entire contents of which are hereby incorporated herein by reference.

### FIELD

The present disclosure relates generally to methods of measuring actinium-225 in biological samples.

### INTRODUCTION

The following paragraphs are not an admission that anything discussed in them is prior art or part of the knowledge of the person of ordinary skill in the art.

Actinium-225 is mainly an anthropogenic alpha emitter from the ²³⁷Np decay series. It has great potential for use in Targeted Alpha Therapy of some types of cancers because it has a relatively short radiological half-life (t_{1/2}) of 10.0±0.1 days and it decays to a series of short live alpha emitters: ²²¹Fr, ²¹⁷At, ²¹³Bi, and ²¹³Po (t_{1/2} of 4.79±0.02 min, 32.3±0.4 ms, 45.59±0.06 min, and 3.70±0.05 μs, respectively). Nuclear workers, researchers, medical staff, and patients can be contaminated with ²²⁵Ac during the production, purification, testing, and use of this isotopes. The committed effective dose (CED) received by people potentially contaminated should be monitored to protect their health.

A preferred method is via urine bioassay samples because it is minimally invasive and the activity concentration of ²²⁵Ac is sufficiently high to be able to detect an internal dose contamination of 1 mSv (0.7 mBql⁻¹, calculated with GenmodPC software which is based on the International Commission of Radiological Protection (ICRP) model)). Bioassay methods to determine the activity concentration of ²²⁵Ac in urine and establish the CED received by a person are needed.

Actinium-225 is usually measured by alpha and gamma spectrometry. Alpha spectrometry may be preferred for a bioassay method because it is more sensitive and therefore more suited to determine trace amounts of ²²⁵Ac. Actinium can be purified from potential interferences using precipitation, solvent extraction, ion exchange chromatography, and extraction chromatography (EXC).

### BRIEF DESCRIPTION OF THE DRAWINGS

The drawings included herewith are for illustrating various examples of apparatuses and methods of the present disclosure and are not intended to limit the scope of what is taught in any way.

FIG. 1 is a schematic representation of methods of the present disclosure.

FIG. 2 shows actinium recovery of urine samples spiked with actinium-225 at different method steps.

FIG. 3 shows actinium recovery as a function of time at a co-precipitation step.

FIG. 4 shows actinium recovery as a function of titanium amount at the co-precipitation step.

FIG. 5 shows actinium measured versus added for spiked samples.

### DETAILED DESCRIPTION

Various apparatuses or methods will be described below to provide an example of an embodiment of each claimed invention. No embodiment described below limits any claimed invention and any claimed invention may cover apparatuses and methods that differ from those described below. The claimed inventions are not limited to apparatuses and methods having all of the features of any one apparatus or method described below, or to features common to multiple or all of the apparatuses or methods described below. It is possible that an apparatus or method described below is not an embodiment of any claimed invention. Any invention disclosed in an apparatus or method described below that is not claimed in this document may be the subject matter of another protective instrument, for example, a continuing patent application, and the applicant(s), inventor(s) and/or owner(s) do not intend to abandon, disclaim or dedicate to the public any such invention by its disclosure in this document.

The teachings described herein relate to new and effective methods to measure actinium-225 in urine samples to establish the effective dose received by persons working with or otherwise exposed to this isotope.

The inventor developed a method to measure ²²⁷Ac in water samples using a DGA (diglycolamide) resin, where Ca(II) was effectively removed prior to the resin separation step using (TiO)₃(PO₄)₂ co-precipitation. Then, Ac(III) was co-precipitated with micrograms of CeF₃ and counted by alpha spectrometry. The inventor recognized that a similar strategy could be used to measure Ac in urine and be more effective than currently available methods because: 1) a (TiO)₃(PO₄)₂ co-precipitation would remove the need to evaporate and digest large volumes of urine; 2) the DGA resin would remove potential matrix and radiological interferences, which would lead to a good alpha resolution; 3) ²²⁷Ac could be used as a recovery tracer for maximum accuracy; and 4) a thin layer of Ac(III) can be more rapidly prepared by co-precipitation with CeF₃ than electrodeposition.

However, urine contains a much larger amount of organic matter than water. When the inventor attempted to directly use the method developed for water to human urine, two main issues were observed: 1) the removal of suspended organic matter before the DGA resin step by filtration was taking several hours; and 2) some soluble organic molecules had a similar behavior to Ac(III) and they were retained and eluted on the DGA resin and then precipitated at the CeF₃ co-precipitation step, which significantly affected the alpha resolution. The inventor demonstrated that KBrO₃ could be used to partially breakdown urine organic matter. By adding a rapid organic matter decomposing step after the (TiO)₃(PO₄)₂ co-precipitation step using KBrO₃ and heat, the (TiO)₃(PO₄)₂/DGA method developed for water can be used for urine and still be an efficient method to determine actinium that is not time consuming.

Accordingly, in an aspect of the present disclosure, a method of determining actinium-225 in a human urine sample can include: preparing the human urine sample to produce a pre-concentrated sample; breaking down at least some organic matter in the pre-concentrated sample to produce a decomposed sample; and purifying the decomposed sample to produce a measurement sample.

In some examples, the preparing step can include co-precipitating Ac in the human urine sample, and separating a first precipitate. The preparing step can include adding a TiCl₃ solution and a H₃PO₄ solution to co-precipitate AcPO₄ with (TiO)₃(PO₄)₂. Between the adding and the separating steps, the preparing step can include waiting at least 15 minutes. The preparing step can include adding a HNO₃ solution for preservation. The preparing step can include adjusting pH to about 3.5. The preparing step can include adding a NaOH solution and/or a HNO₃ solution. The separating step can include centrifuging. The preparing step can include rinsing the first precipitate at least once with a NaCl solution. The preparing step can include dissolving the first precipitate to produce a first solution. The preparing step can include dissolving the first precipitate with a HNO₃ solution and a H₂O₂ solution. After the dissolving step, the preparing step can include separating a first supernate from the first solution as the pre-concentrated sample. The separating step can include centrifuging and/or filtering. The preparing step can include adding actinium-227 as a tracer.

In some examples, the breaking down step can include: adding an oxidative agent to the pre-concentrated sample to produce a second solution; heating the second solution until dryness to produce a first residue; dissolving the first residue to produce a third solution; and separating a second supernate from the third solution as the decomposed sample. The oxidative agent can include KBrO₃. The breaking down step can include dissolving the first residue with a HNO₃ solution and a H₂O₂ solution. Between the heating and the dissolving steps, the removing step can include charring the first residue, and cooling the first residue. The separating step can include centrifuging and/or filtering.

In some examples, the purifying step can include passing the decomposed sample through a purification media. The purification media can include a DGA (diglycolamide) resin. The purifying step can include rinsing the purification media with a HNO₃ solution and a H₂O₂ solution. The purifying step can include eluting actinium from the purification media with a HNO₃ solution to produce a fourth solution.

In some examples, the method can further include: evaporating the fourth solution until dryness to produce a second residue; dissolving the second residue with a HCl solution to produce a fifth solution; micro-precipitating the fifth solution to produce a second precipitate; and separating the second precipitate as the measurement sample. The method can include adding Ce³⁺ and HF to the fifth solution to micro-precipitate AcF₃ with CeF₃. The method can include, between the adding and the separating steps, waiting at least 5 minutes. The separating step can include filtering.

In some examples, the method can further include mounting the measurement sample for counting by alpha spectrometry. The method can include counting the measurement sample by alpha spectrometry.

In an aspect of the present disclosure, a method can include: providing a human urine sample; co-precipitating Ac in the human urine sample to produce a first precipitate; separating the first precipitate; dissolving the first precipitate to produce a first solution; separating a first supernate from the first solution; adding an oxidative agent to the first supernate to produce a second solution; heating the second solution to produce a first residue; dissolving the first residue to produce a third solution; separating a second supernate from the third solution; passing the second supernate through a purification media; eluting actinium from the purification media to produce a fourth solution; evaporating the fourth solution to produce a second residue; dissolving the second residue to produce a fifth solution; micro-precipitating the fifth solution to produce a second precipitate; separating the second precipitate; and mounting the second precipitate for counting by alpha spectrometry.

Other aspects and features of the present disclosure will become apparent upon review of the teachings of the following sections of the detailed description, which are intended to be illustrative but non-limiting.

### I. Experimental

### Reagents and Standards

All solutions used for the present disclosure were prepared using ultrapure water (UPW) from a Millipore Direct-Q5 water purification system (Billerica, MA, USA). Trace metal grade acids (phosphoric acid (H₃PO₄), nitric acid (HNO₃), hydrochloric acid (HCl), hydrofluoric acid (HF)), hydrogen peroxide (H₂O₂), sodium hydroxide (NaOH), sodium chloride (NaCl), potassium bromate (KBrO₃), and cerium nitrate Ce(NO₃)₃ were purchased from Fisher Scientific (Fair Lawn, NJ, USA). Titanium trichloride (TiCl₃) was obtained from Sigma Aldrich (Oakville, ON, Canada). DGA-N® extraction chromatography resin (50-100 μm) pre-packed in 2 ml cartridges, which contains N,N,N′,N′-tetra-n-octyldiglycolamide, was purchased from Eichrom technologies, Inc. (Lisle, IL, USA). Ethanol was obtained from commercial alcohols Inc. (Mississauga, ON, Canada). Certified solutions of ⁹⁰Sr, ²⁰⁹Po, ¹⁴⁷Pm, ²²⁶Ra, ²²⁷Ac, ²³¹Pa, ²²⁹Th, ²³⁷Np, ²³⁹Pu, ²⁴³Am and ²⁴⁴Cm were obtained from Eckert and Ziegler (Valencia, CA, USA). Solutions of natural Th and U were obtained from SCP Science (Baie d′Urfé, QC, Canada). Urine samples were provided by the Chalk River dosimetry laboratory (Chalk River, ON, Canada) from unknown donors.

### Instruments

All alpha spectrometry measurements were performed using an Octete Plus® alpha spectrometer with eight 450 mm² ULTRA-AS ion-implanted silicon detectors (AMETEK/ORTEC Inc., Oak Ridge, TN, USA). For method development, some beta emitters were measured using a Hidex 300 SL liquid scintillation counter (Hidex Oy, Turku, Finland).

### Procedure

The procedure developed to measure ²²⁵Ac in urine had three main steps: 1) sample preparation and Ac pre-concentration; 2) breakdown of organic matter; and 3) purification and CeF₃ micro-precipitation.

### Sample Preparation and Ac Pre-Concentration

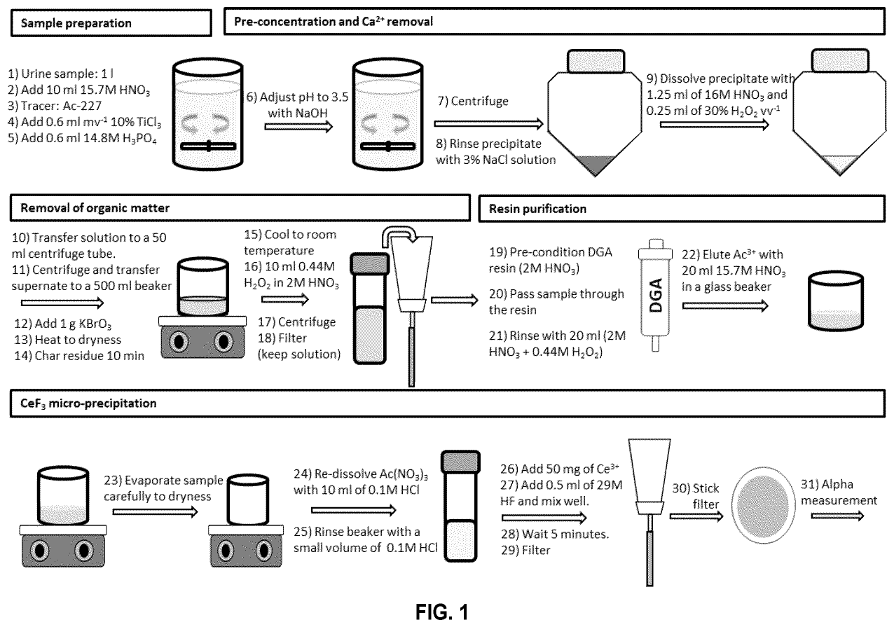

Each urine sample (1 I) was acidified to 1% nitric acid for preservation (FIG. 1, steps 1 and 2). The sample was transferred to a glass beaker. Then, 1 Bq of ²²⁷Ac tracer, 0.6 ml of 14.9M H₃PO₄, and 0.6 ml of 10% TiCl₃ in HCl were added (FIG. 1, steps 3, 4 and 5). The urine sample was mixed and the pH adjusted to 3.5 (FIG. 1, step 6). The (TiO)₃(PO₄)₂ precipitate was recovered by centrifugation and rinsed three times with a 3% NaCl solution (FIG. 1, steps 7 and 8). The precipitate was dissolved with 1.25 ml of 15.7M HNO₃ and 0.25 ml of 30% H₂O₂ w⁻¹ and the solution was transferred to a 50 ml centrifuge tube (FIG. 1, steps 9 and 10). The sample was centrifuged to remove the suspended particles and reduce part of the organic matter. The supernate was transferred to a 500 ml glass beaker (FIG. 1, step 11).

### Breakdown of Organic Matter

To each sample, 1 g of KBrO₃ was added (FIG. 1, step 12). Then, the sample was heated, boiled, and brought to dryness (FIG. 1, steps 13). The dry residue was charred for 10 minutes at 400° C. on the hot plate (FIG. 1, step 14). The beaker was removed from the hot plate and left to cool to room temperature (FIG. 1, step 15). The residue was dissolved with 10 ml of 0.44M H₂O₂ in 2M HNO₃ (FIG. 1, step 16). The sample was transferred to a 50 ml centrifuge tube. The largest particles were removed by centrifugation (FIG. 1, step 17), while the smallest ones were removed using a 50 μm filter (FIG. 1, step 18).

### Purification and CeF₃ Micro-Precipitation

The solution was passed through two 2 ml stacked DGA resin cartridges and the resin was rinsed with 20 ml of 0.44M H₂O₂ in 2M HNO₃ (FIG. 1, steps 19, 20, and 21). Actinium was selectively eluted from the DGA resin with 20 ml of 15.7M HNO₃ (FIG. 1, step 22). The acid solution was evaporated to dryness (FIG. 1, step 23) and the residue dissolved in 10 ml of 0.1M HCl before being transferred to a 50 ml plastic centrifuge tube (FIG. 1, steps 24 and 25). A CeF₃ micro-precipitation was performed and the filter was stuck on a metal disc (FIG. 1, steps 26, 27, 28, 29, and 30). The sample was then counted 48 hours by alpha spectrometry (FIG. 1, step 31).

### Calculations

The net count rate of ²²⁵Ac was indirectly calculated based on the net count rate of ²¹⁷At, because of ²²⁷Th, ²²³Ra, and ²²⁴Ra interferences. There are no interferences for ²¹⁷At peak. ²²⁵Ac decays significantly while counted and therefore the number of counts measured needs to be corrected to what it would be if it did not decay significantly. The net apparent count rate for ²²⁵Ac (r_{225Ac}) (count s⁻¹) was calculated using Equation 1:

$${r}_{22 {5}_{Ac}} = {r}_{21 {7}_{At}} \cdot {λ}_{22 {5}_{Ac}} \cdot {t}_{g} \cdot {e}^{{λ}_{22 {5}_{Ac}} ( {t}_{1} )} / ( 1 - {e}^{- {λ}_{22 {5}_{Ac}} ( {t}_{g} )} ) \qquad ( 1 )$$

where r_{217At} is the net count rate of ²¹⁷At in count s⁻¹, λ_{225Ac} is the decay constant of ²²⁵Ac(Ln(2)/t_{1/2}) in s⁻¹, t_{g} is the counting time in s, t₁ is the time between counting and separation in s. The activity concentration of 225Ac in the urine sample (c_{A}) was calculated using Equation 2:

$${c}_{A} = ( {r}_{22 {5}_{Ac}} \cdot A \cdot b ) / ( {r}_{22 {7}_{Ac}} \cdot V ) \qquad ( 2 )$$

where A is the activity of ²²⁷Ac tracer added in Bq, b is the fraction of ²²⁷Ac that decays through alpha emission (e.g. 0.0142), r_{227Ac} is the net count rate of ²²⁷Ac in count s⁻¹, and V is the sample volume in I (it can also be the mass of sample in kg to express the activity by mass of sample).

### Method Development

Urine samples were spiked at the beginning of the method (FIG. 1, step 3), before the resin separation (FIG. 1, step 20), and before the micro-precipitation step (FIG. 1, step 26) to evaluate at which step Ac recovery losses were more significant. Then, different strategies to improve the chemical recovery at the (TiO)₃(PO₄)₂ co-precipitation and the DGA resin elution steps were tested (FIG. 1, steps 6 and 22, respectively).

(TiO)₃(PO₄)₂ Co-Precipitation Step

Urine samples were spiked with a known amount of ²²⁵Ac. Then, the procedure described above was applied, but with higher amounts of TiCl₃ and longer reaction times at the (TiO)₃(PO₄)₂ co-precipitation step (FIG. 1, step 6).

### DGA Resin Elution

Actinium was dissolved in 10 ml of 2M HNO₃ and the solution passed through a DGA resin. The resin was rinsed with 20 ml of 0.44M H₂O₂ in 2M HNO₃. The load and rinse solutions were combined. Actinium was eluted from the resin with 10 ml of 15.7M HNO₃. However, the elution was done either directly or by small increments of 2.5 and 5 ml. Also, a waiting time of 10 minutes was sometimes added between the fractions eluted. The elution fractions were eluted in the same beaker. The fractions obtained were evaporated to dryness, re-dissolved in 0.1M HCl, and measured by alpha spectrometry after a CeF₃ micro-precipitation.

### Decontamination Factor

The decontamination factor (DF) for potential interferences was determined by spiking urine sample with a known amount of a potential interference: ²³²Th, ²³⁸U, and ²³¹Pa (1 Bq), ²⁰⁹Po, ²²⁶Ra, ²³⁷Np, ²³⁹Pu, ²⁴³Am, and ²⁴⁴Cm (0.1 Bq), ⁹⁰Y (10 Bq), ¹⁴⁷Pm (100 Bq), and Ca (2.5 g). The procedure was applied. The alpha emitters were measured by alpha spectrometry. For the isotopes ⁹⁰Y and ¹⁴⁷Pm, the CeF₃ salt on the filter at the micro-precipitation step (FIG. 1, step 30) was dissolved in 10 ml of 0.1M HCl and the acid solution transferred to an LSC vial. Then, 10 ml of Ultima Gold AB cocktail (PerkinElmer, Guelph, ON, Canada) were added. Yttrium-90 and promethium-147 were measured by LSC. Yttrium-90 was obtained from a ⁹⁰Sr solution after purification with a Sr resin (2 ml cartridges, 50-100 μm, 4,4′ (5′)-di-t-butylcyclohexano 18-crown-6 in 1-octanol) (Eichrom, Lisle, IL). Protactinium solution was dissolved in 12M HCl, passed through an anion exchange resin, and eluted from the resin using 0.1M HCl to remove its progenies. Calcium was determined by gravimetry, by measuring the mass of CaF₂ on the filter.

### Figures of Merit

The minimum detectable activity (MDA) was determined by preparing 10 water blank samples. The procedure was applied and the MDA calculated based on ISO 11929. An equation summarizing the calculation is presented in Equation 3:

$$M D A = \frac{( 2 \cdot ( {k}_{1 - α} \cdot ( A / ( V \cdot {r}_{22 {5}_{Ac}} ) ) \cdot \sqrt{( {r}_{0} / {t}_{g} ) + ( {r}_{0} / {t}_{0} )} ) + ( A / ( V \cdot {r}_{{227}_{Ac}} ) ) \cdot {k}_{1 - α}^{2} / {t}_{g} )}{( 1 - {k}_{1 - α}^{2} \cdot ( {( Δ V / V )}^{2} + {( ( {r}_{22 {7}_{Ac}} / ( b \cdot {t}_{g} ) + ( {r}_{0} / b ) / {t}_{0} ) / {( ( {r}_{22 {7}_{Ac}} - {r}_{0} ) / b )}^{2} + ( Δ A / A ) )}^{2} ) )} \qquad ( 3 )$$

where k_{1-α} is the confidence level constant (e.g. 1.65 for 95%), rₒ the blank count rate of ²¹⁷At in the region of interest of ²¹⁷At, tₒ is the background counting time, and ΔA and ΔV are the relative uncertainty on A and V, respectively. Note that equation 3 symbols were adjusted to correspond to the symbols in Equations 1 and 2.

The method was validated by spiking 10 urine samples with a known amount of ²²⁹Th (²²⁵Ac). The procedure described above was applied and the activity concentration obtained was calculated using Equation 2.

### II. Results and Discussion

### Method Development

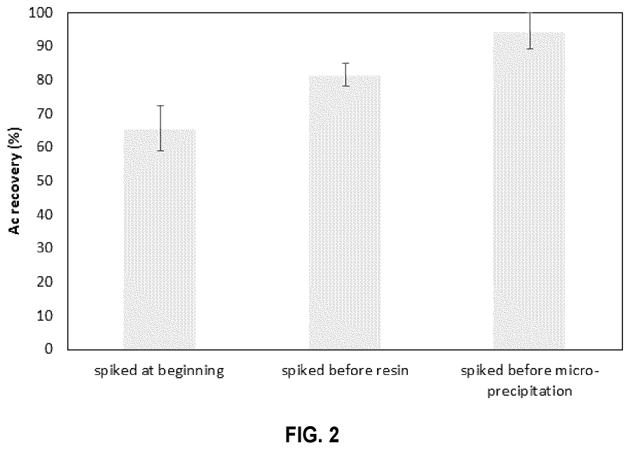

The Ac recovery from urine samples spiked at different steps of the method is shown in FIG. 2. The Ac recovery for the sample spiked at the beginning of the method, before the resin separation, and before the micro-precipitation steps were 66±3%, 82±6%, and 95±5%, respectively. The CeF₃ micro-precipitation was very effective and no improvement was needed for this step. About 15% of the overall Ac added was lost at both the (TiO)₃(PO₄)₂ co-precipitation step and the DGA resin elution step for a total loss of about 30%.

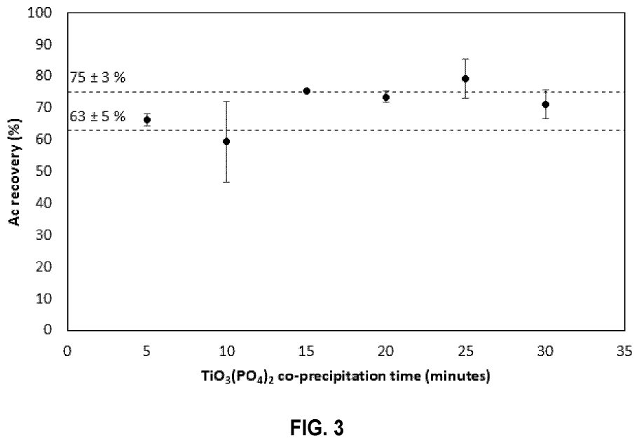

The Ac recovery as a function of the co-precipitation time at the (TiO)₃(PO₄)₂ co-precipitation step (FIG. 1, step 6) is presented in FIG. 3. The recovery was in average 63±5% for the first 10 minutes and then increased to 75±3% for 15 to 30 minutes. Waiting for at least 15 minutes increased the chemical recovery of the method by about 10%. It is why it was decided to wait 15 minutes at this step.

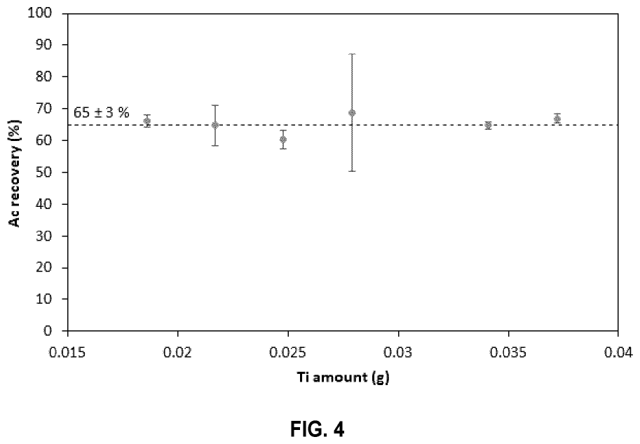

The Ac recovery as a function of the amount of Ti added is shown in FIG. 4. The recovery was in average 65±3% and was constant for the amount of Ti tested. Therefore, the amount of Ti was maintained at 0.019 g. Urine is not a constant matrix like water; thus, the chemical recovery was affected by the different batches of urine used to perform the tests. For each test, the same pooled urine was used, but not between the tests. As a consequence, the chemical recovery value cannot be compared between tests but only relatively to each test.

Actinium was extracted on the DGA resin in 2M HNO₃, rinsed, and eluted with 15.7M HNO₃. Only 1±1% of the Ac was measured in the load and rinse solutions combined (Table 1), which confirmed that Ac was well retained by the resin in these conditions. An Ac recovery of 82±6% was obtained by directly eluting Ac from the resin using 10 ml of 15.7M HNO₃ for the optimization tests (Table 1). A strategy that can be used is to elute the analyte by fraction, thinking that a higher recovery will be obtained. There is no proof to the inventor's knowledge that it makes a difference, but since the eluent to use cannot be changed to obtain a good DF, it was decided to test the idea. An elution by fraction with different waiting times gave recoveries between 73 and 77% (Table 1), which is similar to the regular elution. It was demonstrated previously, where actinides were passed through a TEVA resin under pressure, that a waiting time of 10 minutes could improve the elution. Waiting periods had no effect on Ac recovery in the conditions tested.

TABLE 1
Elution tests (1 DGA resin)

| | | 1^{st} | | | 2^{nd} | | Ac |
|---|---|---|---|---|---|---|---|
| | 1^{st} | waiting | 2^{nd} | | waiting | 3^{rd} | recovery |
| | elution | time | elution | | time | elution | (%) |
| Regular | 10 ml | — | — | | — | — | 82 ± 6 |
| Test 1 | 5 ml | 10 min | 5 | ml | — | — | 77 ± 5 |
| Test 2 | 5 ml | 10 min | 2.5 | ml | 10 min | 2.5 ml | 85 ± 1 |
| Test 3 | — | 10 min | 10 | ml | — | — | 73 ± 14 |
| Load + rinse | | | | | | | 1 ± 1 |

### Decontamination Factor

The DF of various radiological and matrix potential interferences is shown in Table 2. The DF was sufficiently high to ensure that all potential interferences were properly removed from a urine bioassay sample. It was also sufficiently high to remove 229Th, the precursor of ²²⁵Ac tracer. The amount of Ca²⁺ in a 1 I urine sample varies from 30 to 390 mgl⁻¹ according to Putnam study. Calcium was efficiently removed at the (TiO)₃(PO₄)₂ co-precipitation step (FIG. 1, step 6) (DF of 13000±9000) and did not interfere with the alpha resolution at the CeF₃ micro-precipitation step (FIG. 1, step 27). Also, if lanthanides or tri-valent actinides were present, they would not interfere. Note that the DF cannot be seen as an absolute value, because the number of counts measured was extremely low most of the time. It is also why sometimes, the uncertainty on the DF value was very high.

TABLE 2
Potential radiological and matrix interferences

| Isotope to measure | | | Potential interferences | | | |
|---|---|---|---|---|---|---|
| | Energy | Intensity | | Energy | Intensity | Decontamination |
| Isotope | (keV) | (%) | Isotope | (keV) | (%) | factor |
| ²²⁷Ac | 5042 | 0.658 | ²⁰⁹Po | 4977 | 79.2 | ≥3000 ± 2000 |
| | 5029 | 0.546 | | 4979 | 19.8 | |
| | | | ²²⁶Ra | 4871 | 94.0 | ≥4833 ± 3 |
| | | | ²²⁹Th | 4845 | 56.2 | ≥34300 ± 30000 |
| | | | | 4901 | 10.2 | |
| | | | ²³¹Pa | 5104 | 25.3 | ≥1400 ± 400 |
| | | | | 5040 | 22.5 | |
| | | | | 5120 | 20.0 | |
| | | | | 5150 | 11.7 | |
| | | | ²³⁴U | 4858 | 71.4 | ≥35000 ± 25000 |
| | | | ²³⁷Np | 4872 | 47.6 | ≥5470 ± 20 |
| | | | | 4855 | 23.0 | |
| | | | ²⁴²Pu | 4985 | 76.5 | ≥5480 ± 30 |
| | | | | 4940 | 23.4 | |
| | | | ²⁴⁷Cm | 4870 | 71.0 | ≥3000 ± 1000 |
| | | | | 5267 | 13.8 | |
| | | | | 4985 | 2.00 | |
| | | | | 4943 | 1.60 | |
| | | | ²⁴⁸Cm | 5078 | 75.0 | ≥3000 ± 1000 |
| | | | | 5035 | 16.5 | |
| ²¹⁷At | 7200 | 99.9 | — | — | — | — |
| Ca²⁺ | | | | | | ≥13000 ± 9000 |
| Y³⁺ | | | | | | ≥5000 ± 6000 |
| Am³⁺ | | | | 5364 | 86.7 | ≥4960 ± 40 |
| | | | | 5321 | 11.5 | |
| Pm³⁺ | | | | | | ≥600 ± 500 |

### Organic Matter Removal

The organic matter carried by at the (TiO)₂(PO₄) 3 phosphate co-precipitation step was decomposed with KBrO₃ and heat and removed by centrifugation and filtration. The filtration of the sample after decomposition and centrifugation was straightforward and only took a few minutes instead of hours if no decomposition with heat and KBrO₃ was performed. Also, no organic residue was visible on the filter at the CeF₃ micro-precipitation step. The technique used to decompose and remove the organic matter was effective.

### Figures of Merit

### Minimal Detectable Activity and Chemical Recovery

A MDA of 0.2±0.2 mBql⁻¹ was obtained (Table 3), which is lower than the minimum required MDA of 0.7 mBql⁻¹ for a CED of 1 mSv. An average chemical recovery of 62±6% was obtained (Table 3). The method chemical recovery was sufficiently high to meet the required MDA value. A low MDA was obtain, even if the chemical recovery was not particularly high, because the number of background counts in the region of interest of ²¹⁷At was extremely low (result not shown). The chemical recovery was also relatively stable (small standard deviation) for a significant amount of different urine samples tested. Unexpectedly low chemical recovery is very unlikely using this method, which is desired for a bioassay method.

TABLE 3
Figures of merit

| | |
|---|---|
| Average chemical recovery (n = 30) (%) | 62 ± 6 |
| Minimal detectable activity (mBql⁻¹) | 0.2 ± 0.2 |
| MDA needed for 1 mSv | 0.7 |
| (3 days after exposition) (mBql⁻¹) | |
| Mean relative bias (accuracy) (%) | −7.93 |
| Relative precision (precision) (%) | 7.35 |
| Sample per work day/person | 5 |

### Spiked Samples

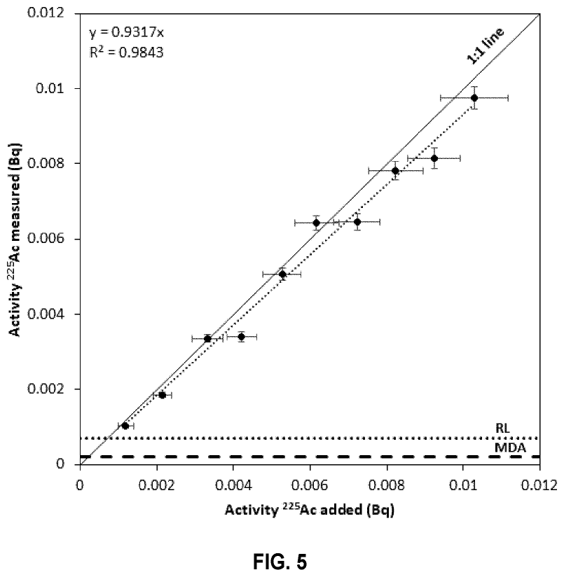

The activity measured as a function of the activity added is shown in FIG. 5, with reference level (RL) and MDA. The activity measured correspond to the activity added. However, there is a mean relative bias of −7.93% (Table 3). The relative precision was 7.35% (Table 3). The mean relative bias and relative precision are within the tolerance values of the ANSI/HPS N13.30 criteria for a valid bioassay method (−25-50% for relative bias and +40% for relative precision). This result demonstrates the validity of the method.

A bias is usually caused when one or both of the standard solution activities used are not exactly as expected (when there is no reason to believe the isotopes could behave differently). It is expected that ²²⁵Ac and ²²⁷Ac had the exact same behavior, since they were both added from standard solutions. The bias obtained could simply be due to the uncertainty on the activity of the standard solution used. Another possible explanation is that ²²⁵Ac and ²¹⁷At did not have the exact same amount of counts on the alpha spectrum, especially when measuring low activity samples (larger uncertainty on the result). Intercomparison with other laboratories and standard solution analysis could help to determine the exact cause of the bias.

### Method Throughput

Five samples can be comfortably performed in one work day using this method by one person (Table 3).

### III. Conclusions

Methods were developed to measure ²²⁵Ac in urine samples. ²²⁵Ac was isolated and purified from potential interferences using (TiO)₃(PO₄)₂ co-precipitation and DGA resin. The method decontamination factors for potential matrix and radiological interferences was high. The overall chemical recovery (62±6%) was optimized and constant. A low MDA of 0.2±0.2 mBql⁻¹ was obtained, which is sufficiently sensitive to determine if a person was contaminated with a CED of more than 1 mSv. The method was validated using spiked samples.

While the above description provides examples of one or more apparatuses or methods, it will be appreciated that other apparatuses or methods may be within the scope of the accompanying claims.

1. A method of determining actinium-225 in a human urine sample, the method comprising:
preparing the human urine sample to produce a pre-concentrated sample;
breaking down at least some organic matter in the pre-concentrated sample to produce a decomposed sample; and
purifying the decomposed sample to produce a measurement sample.

2. The method of claim 1, wherein the preparing step comprises co-precipitating Ac in the human urine sample, and separating a first precipitate.

3. The method of claim 2, wherein the preparing step comprises adding a TiCl₃ solution and a H₃PO₄ solution to co-precipitate AcPO₄ with (TiO)₃(PO₄)₂.

4. (canceled)

5. The method of claim 2, wherein the preparing step comprises adding a HNO₃ solution for preservation.

6. The method of claim 2, wherein the preparing step comprises adjusting pH to about 3.5.

7. The method of claim 6, wherein the preparing step comprises adding a NaOH solution and/or a HNO₃ solution.

8. The method of claim 2, wherein the separating step comprises centrifuging.

9. The method of claim 2, wherein the preparing step comprises rinsing the first precipitate at least once with a NaCl solution.

10. The method of claim 2, wherein the preparing step comprises dissolving the first precipitate to produce a first solution.

11. The method of claim 10, wherein the preparing step comprises dissolving the first precipitate with a HNO₃ solution and a H₂O₂ solution.

12. The method of claim 10, wherein, after the dissolving step, the preparing step comprises separating a first supernate from the first solution as the pre-concentrated sample.

13. The method of claim 12, wherein the separating step comprises centrifuging and/or filtering.

14. The method of claim 1, wherein the preparing step comprises adding actinium-227 as a tracer.

15. The method of claim 1, wherein the breaking down step comprises:
adding an oxidative agent to the pre-concentrated sample to produce a second solution;
heating the second solution until dryness to produce a first residue;
dissolving the first residue to produce a third solution; and
separating a second supernate from the third solution as the decomposed sample.

16. The method of claim 15, wherein the oxidative agent comprises KBrO₃.

17. The method of claim 15, wherein the breaking down step comprises dissolving the first residue with a HNO₃ solution and a H₂O₂ solution.

18. The method of claim 15, wherein, between the heating and the dissolving steps, the breaking down step comprises charring the first residue, and cooling the first residue.

19. The method of claim 15, wherein the separating step comprises centrifuging and/or filtering.

20. The method of claim 1, wherein the purifying step comprises passing the decomposed sample through a purification media, and the purification media comprises a DGA (diglycolamide) resin.

21. (canceled)

22. The method of claim 20, wherein the purifying step comprises rinsing the purification media with a HNO₃ solution and a H₂O₂ solution.

23. The method of claim 20, wherein the purifying step comprises eluting actinium from the purification media with a HNO₃ solution to produce a fourth solution.

24. The method of claim 23, further comprising:
evaporating the fourth solution until dryness to produce a second residue;
dissolving the second residue with a HCl solution to produce a fifth solution;
micro-precipitating the fifth solution to produce a second precipitate; and
separating the second precipitate as the measurement sample.

25. The method of claim 24, comprising adding Ce³⁺ and HF to the fifth solution to micro-precipitate AcF₃ with CeF₃.

26. (canceled)

27. (canceled)

28. (canceled)

29. (canceled)

30. A method, comprising:
providing a human urine sample;
co-precipitating Ac in the human urine sample to produce a first precipitate;
separating the first precipitate;
dissolving the first precipitate to produce a first solution;
separating a first supernate from the first solution;
adding an oxidative agent to the first supernate to produce a second solution;
heating the second solution to produce a first residue;
dissolving the first residue to produce a third solution;
separating a second supernate from the third solution;
passing the second supernate through a purification media;
eluting actinium from the purification media to produce a fourth solution;
evaporating the fourth solution to produce a second residue;
dissolving the second residue to produce a fifth solution;
micro-precipitating the fifth solution to produce a second precipitate;
separating the second precipitate; and
mounting the second precipitate for counting by alpha spectrometry.

31. (canceled)
